(19) United States
(12) Patent Application Publication
(10) Pub. No.: US 2021/0301006 A1
(43) Pub. Date: Sep. 30, 2021

(54) METHODS OF TREATING ACUTE RESPIRATORY DISTRESS SYNDROME

(71) Applicant: FibroGen, Inc, San Francisco, CA (US)

(72) Inventor: Elias KOUCHAKJI, San Francisco, CA (US)

(21) Appl. No.: 17/217,953

(22) Filed: Mar. 30, 2021

Related U.S. Application Data
(60) Provisional application No. 63/001,991, filed on Mar. 30, 2020.

(51) Int. Cl. C07K 16/22; C12N 15/113; A61K 45/06; A61P 11/00

(52) U.S. Cl. CPC C07K 16/22; C12N 15/113; A61K 2039/505; A61P 11/00; A61K 45/06

(57) ABSTRACT

The invention relates to methods and agents useful for treating acute respiratory distress syndrome (ARDS). Methods and agents for treating various physiological and pathological features associated with ARDS are also provided.

### FIELD OF THE INVENTION

The invention relates to methods and agents useful for treating acute respiratory distress syndrome (ARDS). Methods and agents for treating various physiological and pathological features associated with ARDS are also provided.

### BACKGROUND

ARDS is an acute inflammatory pulmonary condition with associated pulmonary edema. The immediate consequences are profound hypoxemia, decreased lung compliance, and increased intrapulmonary shunt and dead space. Even with extreme intervention including ventilation or extracorporeal membrane oxygenation (ECMO) in an intensive care unit (ICU), mortality is high at about 40%-45% (Bellani G, et al. Epidemiology, patterns of care, and mortality for patients with acute respiratory distress syndrome in intensive care units in 50 countries. JAMA. 2016; 315(8):788-800; Li G, et al. Eight-year trend of acute respiratory distress syndrome: a population-based study in Olmsted County, Minn. Am J Respir Crit Care Med. 2011; 183(1):59-66; Villar J, et al. The ALIEN study: incidence and outcome of acute respiratory distress syndrome in the era of lung protective ventilation. Intensive Care Med. 2011; 37(12):1932-41).

ARDS historically extracts a significant toll in the USA with an estimated 190,000 cases annually, resulting in about 74 000 deaths (Rubenfeld G D, et al. Incidence and outcomes of acute lung injury. N Engl J Med 2005; 353:1685-93). Further, ARDS patients require considerable skilled nursing care and account for 10% of admissions to intensive care unit (ICU) and represent 23% of ventilated patients. In addition to the high mortality rate, ARDS survivors suffer from significant muscle weakness and neuropsychiatric problems, such that fewer than 50% return to work within 12 months after leaving intensive care (Herridge M S, et al. One-year outcomes in survivors of the acute respiratory distress syndrome. N Engl J Med. 2003; 348:683-93). The physical, psychological, and cognitive impact of ARDS can markedly impact quality of life for at least five years (Herridge M S, et al. Functional disability 5 years after acute respiratory distress syndrome. N Engl J Med. 2011; 364(14):1293-304), if not longer.

Worryingly, a shocking upsurge in cases of ARDS is occurring worldwide with the spread of SARS-CoV-2, the virus responsible for the disease COVID-19. Based on the experience in Wuhan, China, the epicenter of the disease, about 20% of COVID-19 patients require hospitalization, with 50% of the admitted patients developing hypoxemia by day 8 of admission, of which about 17%-29% develop ARDS. As patient numbers grow and outstrip availability of ventilators and ICU staff, the mortality rate is sadly expected to further increase.

Given the continuing large growth of ARDS patients and the dire clinical course of the syndrome, additional treatments are needed. In particular, treatments that prevent the development of ARDS, lessen the severity of ARDS, reduce the time patients require the use of ventilators, or reduce overall hospitalization times are desperately needed. The present invention meets these needs.

### SUMMARY OF THE INVENTION

In one aspect of the invention, methods are provided for improving, compared to a baseline measurement, the blood oxygenation level of a subject diagnosed with COVID-19. The methods comprise administering to the subject, an effective amount of an anti-CTGF agent, thereby improving the subject's blood oxygenation level. In some embodiments, the subject is further diagnosed with acute respiratory distress syndrome (ARDS). In additional embodiments, the blood oxygenation level is determined using the oxygenation index, the oxygenation saturation index, or pulse oximetry.

In another aspect of the invention, methods are provided for preventing the development of ARDS, or reducing the severity of at least one ARDS-associated symptom or clinical parameter in a subject in need thereof. The methods comprise administering to the subject an effective amount of an anti-CTGF agent, thereby preventing the development of ARDS, or reducing the severity of at least one ARDS-associated symptom or clinical parameter.

In a further aspect of the invention, methods are provided for treating ARDS. The methods comprise administering to a subject in need thereof an effective amount of an anti-CTGF agent, thereby treating the subject's ARDS.

In some embodiments, the subject with ARDS is infected with an infectious agent. In certain embodiments, the infectious agent is a virus. In still further embodiments, the virus is coronavirus selected from the group consisting of SARS-CoV, HCoV NL63, HKU1, MERS-CoV, and SARS-CoV-2. In particular embodiments, the coronavirus is SARS-CoV-2. In additional embodiments, the pulmonary injury suffered by the subject is from a non-infectious agent.

In some embodiments, treatment of a subject with ARDS with an anti-CTGF agent reduces ventilator or ECMO use, reduces time in the ICU or critical care unit (CCU), reduces hospitalization time, reduces all-cause mortality, or reduces supplemental oxygen consumption compared to a control group or historical controls. In other embodiments, treatment of a subject with ARDS with an anti-CTGF agent reduces pulmonary exudate levels or reduces supplemental oxygen consumption compared to a baseline measurement. In further embodiments, treatment of a subject having ARDS with an anti-CTGF agent increases survival compared to a control group or historical controls.

These and other methods of the invention are accomplished by administering an anti-CTGF agent to the subject with ARDS. In some embodiments, the anti-CTGF agent is selected from group consisting of anti-CTGF antibodies, anti-CTGF antibody fragments, anti-CTGF antibody mimetics and anti-CTGF oligonucleotides. In further embodiments, the anti-CTGF agent is an anti-CTGF antibody. In additional embodiments, the anti-CTGF antibody is a human or humanized antibody. In particular embodiments, the anti-CTGF antibody is pamrevlumab. In other embodiments, the anti-CTGF antibody binds to CTGF competitively with pamrevlumab. In a specific embodiment, the anti-CTGF antibody is identical to CLN1 or mAb1 as described in U.S. Pat. No. 7,405,274. In specific embodiments, the effective amount of an anti-CTGF antibody is at least 35 mg/kg. In particular embodiments, at least 35 mg/kg of pamrevlumab is administered on Day 1 and Day 3. In other embodiments, at least 35 mg/kg of pamrevlumab is administered on Day 1 and Day 7.

In some embodiments, the anti-CTGF oligonucleotide is selected from the group consisting of antisense oligonucleotides, siRNAs, miRNAs, and shRNAs. In certain embodiments, the anti-CTGF agent is used in combination with another therapeutic modality. In particular embodiments, the anti-CTGF agent is used in combination with an inhibitor of a pro-inflammatory mediator, an inhibitor of VEGF or an anti-oxidant.

These and other embodiments of the invention will readily occur to those of skill in the art in light of the disclosure herein, and all such embodiments are specifically contemplated. Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### DESCRIPTION OF THE INVENTION

It is to be understood that the invention is not limited to the particular methodologies, protocols, cell lines, assays, and reagents described herein, as these may vary. It is also to be understood that the terminology used herein is intended to describe particular embodiments of the invention, and is in no way intended to limit the scope of the invention as set forth in the appended claims.

It must be noted that as used herein and in the appended claims, the singular forms “a,” “an,” and “the” include plural references unless context clearly dictates otherwise. Thus, for example, a reference to “an anti-CTGF oligonucleotide” includes a plurality of such anti-CTGF oligonucleotides; a reference to “an antibody” is a reference to one or more antibodies and to equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the invention, the preferred methods, devices, and materials are now described. All publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention.

The practice of the invention will employ, unless otherwise indicated, conventional methods of chemistry, biochemistry, molecular biology, cell biology, genetics, immunology and pharmacology, within the skill of the art. Such techniques are explained fully in the literature. See, e.g., Gennaro, A R, ed. Remington's Pharmaceutical Sciences, 18th ed. Mack Publishing Co. (1990); Colowick, S et al., eds., Methods In Enzymology, Academic Press, Inc.; Handbook of Experimental Immunology, Vols. I-IV, DM Weir and CC Blackwell, eds., Blackwell Scientific Publications (1986); Maniatis, T. et al., eds. Molecular Cloning: A Laboratory Manual, 2nd edition, Vols. I-III, Cold Spring Harbor Laboratory Press (1989); Ausubel, F. M. et al., eds. Short Protocols in Molecular Biology, 4th edition, John Wiley & Sons (1999); Ream et al., eds. Molecular Biology Techniques: An Intensive Laboratory Course, Academic Press (1998); PCR (Introduction to Biotechniques Series), 2nd ed. Newton & Graham eds., Springer Verlag (1997).

### Definitions

As used herein, the term “about” refers to ±10% of the numerical value of the number with which it is being used. Therefore, about 50% means in the range of 45%-55%.

Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

As used herein, the term “subject,” “individual,” and “patient” are used interchangeably to refer to a mammal. In a preferred embodiment, the mammal is a primate, and more preferably a human being.

As used herein, the term “acute respiratory distress syndrome” or “ARDS” describes an acute inflammatory syndrome featuring diffuse pulmonary edema and ultimately respiratory failure. The clinicopathological aspects include severe inflammatory injury to the alveolar-capillary barrier, surfactant depletion, and loss of aerated lung tissue. Under the most recent definition, the Berlin definition (Ranieri V M, et al. Acute respiratory distress syndrome: the Berlin Definition. JAMA. 2012; 307(23):2526-33), ARDS is defined by the presence within 7 days of a known clinical insult or new or worsening respiratory symptoms of a combination of acute hypoxemia (PaO₂/FiO₂≤300 mmHg), in a ventilated patient with a positive end-expiratory pressure (PEEP) of at least 5 cmH₂O, and bilateral opacities not fully explained by heart failure or volume overload. The Berlin definition uses the PaO₂/FiO₂ ratio to distinguish mild ARDS (200<PaO₂/FiO₂≤300 mmHg), moderate ARDS (100<PaO₂/FiO₂≤200 mmHg), and severe ARDS (PaO₂/FiO₂≤100 mmHg).

The most widely used means of quantifying ARDS severity relies on a four-point lung injury scoring system (Murray Score or LIS). It is based on the level of PEEP, the ratio of the partial pressure of arterial oxygen (PaO₂) to the fraction of inspired oxygen (FiO₂), the dynamic lung compliance and the degree of radiographic infiltration of pulmonary exudate (Ashbaugh D, et al. Acute respiratory distress in adults. The Lancet. 1967; 290:319-23). Although the LIS has been widely used in clinical studies and a score of >3.0 is commonly used as a qualifying threshold for support with extracorporeal membrane oxygenation (ECMO), it cannot predict outcome during the first 24-72 hours of ARDS (Bernard G R, et al. The American-European consensus Conference on ARDS. definitions, mechanisms, relevant outcomes, and clinical trial coordination. Am J Respir Crit Care Med. 1994; 149:818-24). When the scoring system is used 4-7 days after the onset of the syndrome, scores of 2.5 or higher predicted a complicated course requiring prolonged mechanical ventilation (Ferguson N D, et al. The Berlin definition of ARDS: an expanded rationale, justification, and supplementary material. Intensive Care Med. 2012; 38:1573-82).

ARDS results from acute inflammation affecting the lung's gas exchange surface, the alveolar-capillary membrane (Fanelli V, Ranieri V M. Mechanisms and clinical consequences of acute lung injury. Ann Am Thorac Soc. 2015; 12(Supplement 1):S3-S8). The acute inflammation produces high permeability pulmonary edema with associated recruitment of neutrophils and other mediators of inflammation. The resulting acute inflammatory exudate inactivates surfactant leading to collapse and consolidation of distal airspaces with progressive loss of the lung's gas exchange surface area. This would be compensated for by hypoxic pulmonary vasoconstriction, if the inflammatory process did not also effectively paralyse the lung's means of controlling vascular tone, thereby allowing deoxygenated blood to cross unventilated lung units on its way to the left heart. The combination of these two processes causes profound hypoxemia and eventually type 2 respiratory failure as hyperventilation fails to keep pace with carbon dioxide production (Griffiths M J D, et al. Guidelines on the management of acute respiratory distress syndrome BMJ Open Respiratory Research 2019; 6:e000420. doi: 10.1136/bmjresp-2019-000420).

The terms, “treat,” “treating” and “treatment,” as used herein, refer to the administration of a therapeutic agent (e.g., anti-CTGF agent) to a subject in need thereof, in order to achieve a beneficial effect including changes in a pathological feature of a cell type, tissue or organ affected by infection with SARS-CoV-19 or by ARDS; the prevention or reduction of one or more symptoms of ARDS; improvement in a subject's prognosis; or an increase in the likelihood of a subject's survival. Treating a subject afflicted with a disease or condition associated with the development of ARDS, e.g., a bacterial or viral infection, before the development of ARDS, may prevent the development of ARDS, or reduce the severity of ARDS that may develop later. Treatment, before or after the development of ARDS may also reduce the need for medication or supportive measures, such as intubation and ventilation, or ECMO.

In some embodiments, treatment of subjects with ARDS or with a likelihood of developing ARDS using an effective amount of an anti-CTGF agent increases the survival of the subjects. In further embodiments, the administration of an effective amount of an anti-CTGF agent increases the survival of subjects with ARDS by at least 10%, 20%, 30%, 40%, 50%, 60%, 70%, 80%, 90% or 100% compared to a control group or historical controls. In other embodiments, treatment with an effective amount of an anti-CTGF agent increases survival by at least 7 days, 14 days, 21 days, 28 days, 1 month, 2 months, 4 months, 6 months, or 12 months compared to a control group or historical controls. In additional embodiments, survival is measured after 1 week, 2 weeks, 3 weeks, 4 weeks, 30 days, 60 days, or 1 year post-hospitalization or post-intubation.

In other embodiments, the administration of an anti-CTGF agent reduces the time needed on a ventilator by an ARDS patient, measured from time of intubation. In particular embodiments, administration of an anti-CTGF agent reduces the time needed on a ventilator by an ARDS patient, by at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, or at least 4 weeks compared to a control group or historical controls. In further embodiments, treatment with an anti-CTGF agent reduces the need for mechanical ventilation at least 5%, 10%, 15%, 20%, 25%, 30%, 35%, 40%, 45%, 50%, 55%, 60%, 65%, 70%, 75%, 80%, 90%, 95%, or 100% compared to a matched control group or historical controls. In other embodiments, treatment with an anti-CTGF agent increased the proportion of subjects that never require mechanical ventilation by at least 5%, 10%, 15%, 20%, 25%, 30%, 35%, 40%, 45%, 50%, 55%, 60%, 65%, 70%, 75%, 80%, 90%, 95%, or 100% compared to a matched control group or historical controls.

In some embodiments, administration of anti-CTGF agents, reduces the need for ECMO, compared to a matched control group or historical controls. In particular embodiments, treatment with an anti-CTGF agent reduces the need for ECMO by at least 5%, 10%, 15%, 20%, 25%, 30%, 35%, 40%, 45%, 50%, 55%, 60%, 65%, 70%, 75%, 80%, 90%, 95%, or 100% compared to a matched control group or historical controls. In other embodiments, treatment with an anti-CTGF agent reduces the days on mechanical ventilation or ECMO by at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days compared to a matched control group or historical controls.

As used herein, the terms “reduce,” “reducing,” or “reduction” in the context of treating a subject with ARDS refers to treatment that eases, mitigates, alleviates, ameliorate or decreases the effect or severity of a symptom of ARDS, without curing the underlying disease, e.g., SARS-CoV-2 infection. Any indicia of success in reducing one or more symptoms of ARDS is recognized as reducing the symptoms. The reduction of an ARDS symptom can be determined using standard routine clinical tests, radiologic or other imaging modalities and observations including ventilator settings, blood oxygenation levels, supplemental oxygen consumption that are well within the skill and knowledge of a medical professional. Other exemplary measurements or tests that can be used to monitor response to treatment include a reduced need for medications such as vasopressors, or the resolution of leukopenia.

In some embodiments, the methods of the invention reduce the occurrence or severity of an ARDS symptom by at least 10%, at least 15%, at least 20%, at least 25%, at least 30%, at least 35%, at least 40%, at least 50%, at least 60%, at least 70%, at least 80%, or at least 90% compared to a baseline measurement or to a control group or historical controls.

In some embodiments, the administration of an effective amount of anti-CTGF agent to a subject with ARDS reduces the extent of pulmonary exudate compared to a baseline measurement. Typically, the occurrence and extent of pulmonary exudate is determined using radiographic imaging techniques, such as chest x-rays or CT scans, but any suitable modality can be used. In some embodiments, the administration of an anti-CTGF agent reduces the extent of pulmonary exudate by at 10%, at least 15%, at least 20%, at least 25%, at least 30%, at least 35%, at least 40%, at least 50%, at least 60%, at least 70%, at least 80%, or at least 90% compared to a baseline measurement.

In some embodiments, a method is provided for increasing, compared to a baseline measurement, the blood oxygenation level in a subject with ARDS. The method comprises administering to the subject an anti-CTGF antibody, thereby increasing the subject's blood oxygenation level. In particular embodiments, blood oxygenation status is expressed as the ration PaO₂/FiO₂, where:

PaO₂=Partial pressure of oxygen in arterial blood, in mmHg; and

FiO₂=Fraction of inspired oxygen, in percent.

In further embodiments, the blood oxygenation level is measured using the oxygenation index (OI), calculated as follows:

OI=[FiO₂×mean airway pressure×100)/PaO₂], and

mean airway pressure is measured in mmHg.

The OI measures the fraction of inspired oxygen (FiO₂) and its usage within the body. A lower oxygenation index is better. As the oxygenation of a person improves, a higher PaO₂ will be achieved at a lower FiO₂, thus lowering the measured OI.

In other embodiments, the blood oxygenation level is measured using the oxygenation saturation index (OSI), calculated as follows:

OSI=[FiO₂×mean airway pressure×100)/oxygen saturation by pulse oximetry (SpO₂)]

The OSI is a reliable noninvasive surrogate for the OI that is associated with hospital mortality and ventilator-free days (VFDs) in patients with ARDS (DesPrez K, et al. Oxygenation Saturation Index Predicts Clinical Outcomes in ARDS. Chest. 2017 December; 152(6): 1151-1158).

In further embodiments, the blood oxygenation level is determined using pulse oximetry. This method measures peripheral oxygen saturation (SpO₂), typically measured using a subject's finger, ear, or toe.

In some embodiments, administration of anti-CTGF agents according to the methods of the invention, increases a subject's blood oxygenation level, as measured by PaO₂/FiO₂, OI, OSI, SpO₂ or other suitable means, at least 5%, 10%, 15%, 20%, 25%, 30%, 35%, 40%, 45%, 50%, 55%, 60%, 65%, 70%, 75%, 80%, 90%, 95%, or 100% compared to a baseline measurement taken before the administration of the anti-CTGF agent. In further embodiments, the comparison measurement is made about 24 h, 36 h, 48 h, 72 h, 96 h, 108 h, 120 h, 132 h, 148 h, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, or 24 days following initiation of anti-CTGF agent therapy.

In other embodiments, administration of anti-CTGF agents according to the methods of the invention, increases a subject's blood oxygenation level, as measured by PaO₂/FiO₂, OI, OSI, SpO₂ or other suitable means, at least 5%, 10%, 15%, 20%, 25%, 30%, 35%, 40%, 45%, 50%, 55%, 60%, 65%, 70%, 75%, 80%, 90%, 95% or 100% compared to a match control group or historical controls. The blood oxygenation level measurement can be taken approximately 24 h, 36 h, 48 h, 72 h, 96 h, 108 h, 120 h, 132 h, 148 h, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days or 28 days following initial diagnosis of ARDS, initiation of treatment, or intubation for mechanical ventilation.

As used herein, the term “effective amount” in the context of administering an anti-CTGF agent to a subject with an ARDS or with the likelihood of developing ARDS, refers to the amount of an anti-CTGF agent that is sufficient to produce a beneficial or therapeutic effect including: the prevention of ARDS; the stabilization of one or more symptoms of the ARDS; the amelioration or reduction in the severity of one or more symptoms of ARDS; an improvement in clinical status; or an increase in survival. In some embodiments, an “effective amount” of an anti-CTGF agent refers to an amount of the anti-CTGF agent that is sufficient to prevent the development of hypoxia, dyspenia, or other clinical symptoms of COVID-19. In further embodiments, the administration of an anti-CTGF agent according to the methods of the invention prevent the further decline, slow the decline, or reverse the decline in blood oxygenation levels in a subject at risk for developing ARDS. In additional embodiments, the anti-CTGF agent is administered to a subject at risk for developing ARDS when the subject's blood oxygenation level, typically measured by pulse oximetry although any suitable means of measurement may be used, falls to below about 99%, 98%, 97%, 96%, 95%, 94%, 93%, 92%, 91%, 90%, 89%, 88%, 87%, 86%, or 85%.

Other clinical parameters that can be used to measure the health status of a subject with ARDS include serum creatinine (kidney function), serum bilirubin (liver function), platelet count (hematologic system function), Glasgow Coma Scale score (neurologic function), or the use of vasopressors (cardiovascular function).

In some embodiments, the clinical status of patients is assessed using an 8-point Modified WHO Ordinal Scale as specified below:

1. Not hospitalized, no limitations on activities

2. Not hospitalized, limitations on activities and/or requiring home oxygen

3. Hospitalized, not requiring supplemental oxygen, no longer requiring medical care

4. Hospitalized, not requiring supplemental oxygen, requiring ongoing medical care (COVID-19-related or otherwise)

5. Hospitalized, requiring supplemental oxygen

6. Hospitalized, requiring nasal high-flow oxygen, non-invasive mechanical ventilation, or both

7. Hospitalized, requiring invasive mechanical ventilation, extra-corporeal membrane oxygenation (ECMO), or both

8. Death

In some embodiments, treatment of subjects with ARDS with an anti-CTGF antibody improves their clinical status by at least 1 point, using the Modified WHO Ordinal Scale, over a matched control group or historical controls.

In other embodiments, treatment of subjects with ARDS with an anti-CTGF antibody hastens their recovery time compared to a matched control group or historical controls. Recovery time is defined as the first day on which a patient satisfies one of the following three categories from the ordinal scale: hospitalized, not requiring supplemental oxygen; not hospitalized (discharged), but with limitation on activities and/or requiring home oxygen; or not hospitalized (discharged), with no limitations on activities and not requiring supplemental oxygen. In additional embodiments, treatment of a subject with ARDS with an anti-CTGF antibody hastens the subject's recovery time, compared to a matched control group or historical controls, by at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days.

In other embodiments, treatment of subjects with ARDS with an anti-CTGF antibody reduces all-cause mortality by at least 5%, 10%, 15%, 20%, 25%, 30%, 35%, 40%, 45%, 50%, 55%, 60%, 65%, 70%, 75%, 80%, 90%, 95%, or 100% compared to a matched control group or historical controls. In further embodiments, treatment of subjects with ARDS with an anti-CTGF antibody reduces time in the ICU or CCU by at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, or 28 days compared to a matched control group or historical controls. In additional embodiments, treatment of subjects with ARDS with an anti-CTGF antibody reduces hospitalization time by at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, or 28 days compared to a matched control group or historical controls. In other embodiments, treatment of subjects with ARDS with an anti-CTGF antibody reduces supplemental oxygen consumption at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, or 28 days compared to a matched control group or historical controls.

### Subjects

In some embodiments, subjects suitable for or in need of treatment with anti-CTGF agents according to the methods of the present invention are mammals, more preferably humans, who are at risk of developing ARDS, are experiencing hypoxia or dyspenia, or have already displayed at least one symptom of ARDS. Subjects at risk for developing ARDS include subjects with a diagnosis of a bacterial or virological infection, including pulmonary infection. Typically, diagnosis is made by polymerase chain reaction (PCR) or culturing of a sample obtained, for example, from the subject's nasal passages, throat, mouth, sinuses, lungs, sputum, saliva or blood. Infectious agents associated with the development of ARDS include bacteria, particularly pneumococcia, mycoplasmas, and protozoans, and viruses, particularly, respiratory viruses that cause nosocomial or community-acquired viral pneumonia, including the Herpesviridae members herpes simplex virus (HSV) and cytomegalovirus (CMV). Other viruses associated with the development of ARDS include members of Coronaviridae, and, more specifically, members of the sub-family Orthocoronavirinae, also known as coronaviruses. Particular coronaviruses associated with the development of ARDS include SARS-CoV, HCoV NL63, HKU1, MERS-CoV, and SARS-CoV-2.

In some embodiments, a method of preventing ARDS is provided, the method comprising administering to a subject at risk of developing ARDS an effective amount of an anti-CTGF agent, thereby preventing the development of ARDS. In particular embodiments, a method of preventing the development of ARDS in a subject with a SARS-CoV-2 infection is provided, the method comprises administering to the infected subject an effective amount of an anti-CTGF agent, thereby preventing the development of ARDS in the subject. In further embodiments, a method of treating ARDS associated with an infection with SARS-CoV-2 is provided, the method comprises administering to a subject with ARDS, an effective amount of an anti-CTGF agent, thereby treating the subject's ARDS.

ARDS may also develop as a consequence of sepsis, septic shock, or toxic shock following a non-pulmonary infection with a bacterium, fungus, protozoan or virus. Common non-pulmonary organ locations of the primary infection include brain, skin, urinary track and abdominal organs. More than 50% of cases of sepsis are the result of an infection with a gram-positive bacteria, most commonly staphylococci. Other commonly implicated bacteria include Streptococcus pyogenes, Escherichia coli, Pseudomonas aeruginosa, and Klebsiella species. Fungal sepsis accounts for approximately 5% of severe sepsis and septic shock cases; the most common cause of fungal sepsis is an infection by Candida species of yeast. In some embodiments, a method of preventing the development of ARDS in a subject with sepsis, septic shock or toxic shock is provided. The method comprises administering to the subject with sepsis, septic shock, or toxic shock an effective amount of an anti-CTGF agent, thereby preventing the development of ARDS. In further embodiments, a method of treating a subject with ARDS associated with sepsis, septic shock, or toxic shock is provided. The method comprises administering to the subject with ARDS associated with sepsis, septic shock, or toxic shock an effective amount of an anti-CTGF agent, thereby treating the subject's ARDS.

Additional subjects at risk for developing ARDS include patients that experience various pulmonary injuries caused by non-infectious agents or factors, such as aspiration of gastric contents, near-drowning, blunt chest contusion, multiple injuries, inhalation burns, pancreatitis, and multiple blood transfusions. The risk of developing ARDS further increases when, in addition to any of the above listed causes, the subject also has advanced age (≥60 years), the presence of hypertension, diabetes, or other comorbidities. Subjects suspected of having ARDS can be readily identified by any competent medical practitioner using standard diagnostic tests and criteria including blood tests or radiographic imaging.

Patient characteristics associated with the likelihood of requiring ventilation include elevated lactate dehydrogenase, elevated high-sensitivity C-reactive protein, elevated interleukin-6, elevated D-dimer, and chest radiographic abnormalities (Wu C et al. JAMA Intern Med 2020 Mar. 13).

Agents

In any of the methods described above, it is particularly contemplated that the agent or medicament that inhibits CTGF (i.e., the anti-CTGF agent or medicament) may be a polypeptide, polynucleotide, or small molecule; for example, an antibody that binds to CTGF, a CTGF antisense molecule, miRNA, ribozyme or siRNA, a small molecule chemical compound, etc. In some embodiments, inhibition of CTGF is accomplished using an antibody that specifically binds CTGF. In further embodiments, the invention contemplates inhibiting CTGF using an anti-CTGF oligonucleotide, but inhibition of CTGF can be accomplished by any of the means well-known in the art for modulating the expression or activity of CTGF.

### Antibodies

The term “antibody” is used in the broadest sense and specifically covers monoclonal antibodies (including full length monoclonal antibodies), polyclonal antibodies, multispecific antibodies (e.g., bispecific antibodies), and antibody fragments, so long as they exhibit the desired biological activity. The term antibody further includes antibody mimetics, discussed further below.

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler G and Milstein C, Nature, 256:495-497 (1975); Harlow E et al., Antibodies: A Laboratory Manual (Cold Spring Harbor Laboratory Press, 2nd ed. (1988); recombinant DNA methods (see, e.g., U.S. Pat. No. 4,816,567); phage-display technologies (see, e.g., Clackson T et al., Nature, 352: 624-628 (1991); Marks J D et al., J Mol Biol 222: 581-597 (1992); and Lee V et al., J Immunol Methods 284(1-2): 119-132 (2004)), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits A et al., Proc Natl Acad Sci USA 90: 2551 (1993); U.S. Pat. Nos. 5,545,807; 5,545,806; 5,569,825; 5,625,126; 5,633,425; and 5,661,016).

Monoclonal antibodies specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass (see, e.g., U.S. Pat. No. 4,816,567; and Morrison S et al., Proc Natl Acad Sci USA 81:6851-6855 (1984)).

“Humanized” forms of non-human (e.g., murine) antibodies are chimeric antibodies that contain minimal sequence derived from non-human immunoglobulin. In some embodiments, a humanized antibody is a human immunoglobulin (recipient antibody) in which residues from a one or more hypervariable regions (HVRs) of the recipient are replaced by residues from one or more HVRs of a non-human species (donor antibody) such as mouse, rat, rabbit, or nonhuman primate having the desired specificity, affinity, and/or capacity. For further details, see, e.g., Jones T A et al., Nature 321:522-525 (1986); Riechmann L et al., Nature 332:323-329 (1988); and U.S. Pat. Nos. 6,982,321 and 7,087,409.

A “human antibody” is one which possesses an amino acid sequence which corresponds to that of an antibody produced by a human and/or has been made using any of the techniques for making human antibodies (see e.g., Hoogenboom H R and Winter G, J. Mol. Biol., 227:381 (1992); Marks J D et al., J. Mol. Biol., 222:581 (1991); Boerner R et al., J. Immunol., 147(1):86-95 (1991); Li J et al., Proc. Natl. Acad. Sci. USA, 103:3557-3562 (2006) and U.S. Pat. Nos. 6,075,181 and 6,150,584).

The anti-CTGF antibodies of the invention may be specific for CTGF endogenous to the species of the subject to be treated or may be cross-reactive with CTGF from one or more other species. In some embodiments, the antibody for use in the present methods is obtained from the same species as the subject in need. In other embodiments, the antibody is a chimeric antibody wherein the constant domains are obtained from the same species as the subject in need and the variable domains are obtained from another species. For example, in treating a human subject, the antibody for use in the present methods may be a chimeric antibody having constant domains that are human in origin and variable domains that are mouse in origin. In preferred embodiments, the antibody for use in the present methods binds specifically to the CTGF endogenous to the species of the subject in need. Thus, in certain embodiments, the antibody is a human or humanized antibody, particularly a monoclonal antibody, that specifically binds human CTGF (GenBank Accession No. NP_001892).

Exemplary antibodies for use in the treatment methods of the present invention are described, e.g., in U.S. Pat. No. 5,408,040; PCT/US1998/016423; PCT/US1999/029652; International Publication No. WO 99/33878; U.S. Pat. No. 9,587,015; WO 2013/108869; and, Myzithras et al. Bioanalysis. 2018 Mar. 1; 10(6):397-406. Preferably, the anti-CTGF antibody is a monoclonal antibody. Also preferably, the antibody is a neutralizing antibody. In particular embodiments, the antibody is the antibody described and claimed in U.S. Pat. Nos. 7,405,274 and 7,871,617. In some embodiments, the antibody for prevention or treatment of ARDS has the amino acid sequence of the antibody produced by the cell line identified by ATCC Accession No. PTA-6006. In other embodiments, the antibody binds to CTGF competitively with an antibody produced by ATCC Accession No. PTA-6006. In certain embodiments, the anti-CTGF antibody binds to domain 2 of human CTGF. In further embodiments, the antibody binds to the same epitope as the antibody produced by ATCC Accession No. PTA-6006. A particular antibody for use in the disclosed treatment methods is CLN1 or mAb1 as described in U.S. Pat. No. 7,405,274, or an antibody substantially equivalent thereto or derived therefrom. In some embodiments, the anti-CTGF antibody is CLN1, an antibody identical to the antibody produced by the cell line identified by ATCC Accession No. PTA-6006 that is encompassed by the claims of U.S. Pat. Nos. 7,405,274 and 7,871,617. In specific embodiments, the anti-CTGF antibody is pamrevlumab (CAS #946415-13-0).

As referred to herein, the phrase “an antibody that specifically binds to CTGF” includes any antibody that binds to CTGF with high affinity. Affinity can be calculated from the following equation:

$$Affinity = {K}_{a} = \frac{[ Ab \cdot Ag ]}{[ Ab ] [ Ag ]} = \frac{1}{{K}_{d}}$$

where [Ab] is the concentration of the free antigen binding site on the antibody, [Ag] is the concentration of the free antigen, [Ab·Ag] is the concentration of occupied antigen binding sites, Kₐ is the association constant of the complex of antigen with antigen binding site, and K_{d} is the dissociation constant of the complex. A high-affinity antibody typically has an affinity at least on the order of 10⁸ M⁻¹, 10⁹M⁻¹ or 10¹⁰ M⁻¹. In particular embodiments, an antibody for use in the present methods will have a binding affinity for CTGF between of 10⁸ M⁻¹ and 10¹⁰ M⁻¹, between 10⁸ M⁻¹ and 10⁹ M⁻¹ or between 10⁹M⁻¹ and 10¹⁰ M⁻¹. In some embodiments the high-affinity antibody has an affinity of about 10⁸ M⁻¹, 10⁹M⁻¹ or 10¹⁰ M⁻¹.

“Antibody fragments” comprise a functional fragment or portion of an intact antibody, preferably comprising an antigen binding region thereof. A functional fragment of an antibody will be a fragment with similar (not necessarily identical) specificity and affinity to the antibody which it is derived. Non-limiting examples of antibody fragments include Fab, F(ab)₂, and Fv fragments that can be produced through enzymatic digestion of whole antibodies, e.g., digestion with papain, to produce Fab fragments. Other non-limiting examples include engineered antibody fragments such as diabodies (Holliger P et al. Proc Natl Acad Sci USA, 90: 6444-6448 (1993)); linear antibodies (Zapata G et al. Protein Eng, 8(10):1057-1062 (1995)); single-chain antibody molecules (Bird K D et al. Science, 242: 423-426 (1988)); single domain antibodies, also known as nanobodies (Ghahoudi M A et al. FEBS Lett. 414: 521-526, (1997)); domain antibodies (Ward E S et al. Nature. 341: 544-546, (1989)); and multispecific antibodies formed from antibody fragments.

### Antibody Mimetics

Antibody mimetics are proteins, typically in the range of 3-25 kD, that are designed to bind an antigen with high specificity and affinity like an antibody, but are structurally unrelated to antibodies. Frequently, antibody mimetics are based on a structural motif or scaffold that can be found as a single or repeated domain from a larger biomolecule. Examples of domain-derived antibody mimetics include AdNectins that utilize the 10th fibronectin III domain (Lipovšek D. Protein Eng Des Sel, 24:3-9 (2010)); Affibodies that utilize the Z domain of staphylococcal protein A (Nord K et al. Nat Biotechnol. 15: 772-777 (1997)), and DARPins that utilize the consensus ankyrin repeat domain (Amstutz P. Protein Eng Des Sel. 19:219-229 (2006)). Alternatively, antibody mimetics can also be based on the entire structure of a smaller biomolecule, such as Anticalins that utilize the lipocalin structure (Beste G et al. Proc Natl Acad Sci USA. 5:1898-1903 (1999)). In some embodiments, the anti-CTGF antibody is an antibody mimetic.

### Oligonucleotides

In some aspects, the present invention comprises synthetic oligonucleotides that decrease the expression of human CTGF mRNA. These anti-CTGF oligonucleotides include isolated nucleic acids, nucleic acid mimetics, and combinations thereof. Oligonucleotides of the invention comprise antisense oligonucleotides, ribozymes, external guide sequence (EGS) oligonucleotides (oligozymes) and inhibitory RNA (RNAi) including siRNA, microRNA (miRNA), and short hairpin RNA (shRNA). Oligonucleotides that decrease the expression of CTGF mRNA are useful for treating ARDS.

The terms “oligonucleotide” and “oligomeric nucleic acid” refer to oligomers or polymers of ribonucleic acid (RNA), deoxyribonucleic acid (DNA), mimetics or analogs of RNA or DNA, or combinations thereof, in either single- or double-stranded form. Oligonucleotides are molecules formed by the covalent linkage of two or more nucleotides or their analogs.

The terms “complementary” and “complementarity” refer to conventional Watson-Crick base-pairing of nucleic acids. For example, in DNA complementarity, guanine forms a base pair with cytosine and adenine forms a base pair with thymine, whereas in RNA complementarity, guanine forms a base pair with cytosine, but adenine forms a base pair with uracil in place of thymine. An oligonucleotide is complementary to a RNA or DNA sequence when the nucleotides of the oligonucleotide are capable of forming hydrogen bonds with a sufficient number of nucleotides in the corresponding RNA or DNA sequence to allow the oligonucleotide to hybridize with the RNA or DNA sequence. In some embodiments, the oligonucleotides have perfect complementarity to human CTGF mRNA, i.e., no mismatches.

When used in the context of an oligonucleotide, “modified” or “modification” refers to an oligonucleotide that incorporates one or more unnatural (modified) sugar, nucleobase or internucleoside linkage. Modified oligonucleotides are structurally distinguishable, but functionally interchangeable with naturally occurring or synthetic unmodified oligonucleotides and usually have enhanced properties such as increased resistance to degradation by exonucleases and endonucleases, or increased binding affinity. In some embodiments, the anti-CTGF oligonucleotides are modified.

Unnatural covalent internucleoside linkages, i.e., modified backbones, include those linkages that retain a phosphorus atom in the backbone and also those that do not have a phosphorus atom in the backbone. Numerous phosphorous containing modified oligonucleotide backbones are known in the art and include, for example, phosphoramidites, phosphorodiamidate morpholinos, phosphorothioates, phosphorodithioates, phosphotriesters, aminoalkylphosphotri-esters, methyl and other alkyl phosphonates including 3′-alkylene phosphonates, 5′-alkylene phosphonates and chiral phosphonates, and phosphinates. See Swayze E and Bhat B in Antisense Drug Technology Principles, Strategies, and Applications. 2nd Ed. CRC Press, Boca Rotan Fla., p. 144-182 (2008).

In further embodiments, the unnatural internucleoside linkages are uncharged and, in others, the linkages are achiral. In some embodiments, the unnatural internucleoside linkages are uncharged and achiral, e.g., peptide nucleic acids (PNAs).

In some embodiments, the modified sugar moiety is a sugar other than ribose or deoxyribose. In certain embodiments, the sugar is arabinose, xylulose, or hexose. In further embodiments, the sugar is substituted with one of the following at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C1 to C10 alkyl or C2 to C10 alkenyl and alkynyl. In some embodiments, the modifications include 2′-methoxy (2′-O—CH3), 2′-aminopropoxy (2′-OCH2CH2CH2NH2), 2′-allyl(2′-CH2-CH═CH2), 2′-O-allyl (2′-O-CH2-CH═CH2) and 2′-fluoro (2′-F). The 2′-modification may be in the arabino (up) position or ribo (down) position. Similar modifications may also be made at other positions on an oligonucleotide, particularly the 3′ position of the sugar on the 3′ terminal nucleotide or in 2′-5′ linked oligonucleotides and the 5′ position of 5′ terminal nucleotide.

In some embodiments, the modified sugar is conformationally restricted. In further embodiments, the conformational restriction is the result of the sugar possessing a bicyclic moiety. In other embodiments, the bicyclic moiety links the 2′-oxygen and the 3′ or 4′-carbon atoms. In additional embodiments the linkage is a methylene (—CH2-)n group bridging the 2′ oxygen atom and the 4′ carbon atom, wherein n is 1 or 2. This type of structural arrangement produces what are known as “locked nucleic acids” (LNAs). See Koshkin A A et al. Tetrahedron, 54, 3607-3630 (1998); and Singh S K et al., Chem. Commun, 4:455-456 (1998).

In some embodiments, the sugar is a sugar mimetic that is conformationally restricted, resulting in a conformationally constrained monomer. In certain embodiments, the sugar mimetic comprises a cyclohexyl ring that comprises one ring heteroatom and a bridge making the ring system bicyclic. See PCT/US2010/044549. In further embodiments, the oligonucleotides comprise at least one nucleotide that has a bicyclic sugar moiety or is otherwise conformationally restricted.

In some embodiments, the modified sugar moiety is a sugar mimetic that comprises a morpholino ring. In further embodiments, the phosphodiester internucleoside linkage is replaced with an uncharged phosphorodiamidate linkage. See Summerton J and Weller D, Antisense Nucleic Acid Drug Dev, 7: 187-195 (1997).

In some embodiments, both the phosphate groups and the sugar moieties are replaced with a polyamide backbone comprised of repeating N-(2-aminoethyl)-glycine units to which the nucleobases are attached via methylene carbonyl linkers. These constructs are called peptide nucleic acids (PNAs). PNAs are achiral, uncharged, and, because of the peptide bonds, resistant to endo- and exonucleases. See Nielsen P E et al., Science, 254:1497-1500 (1991) and U.S. Pat. No. 5,539,082.

Oligonucleotides useful in the methods of the invention include those comprising entirely or partially of naturally occurring nucleobases. Naturally occurring nucleobases include adenine, guanine, thymine, cytosine, uracil, 5-methylcytidine, pseudouridine, dihydrouridine, inosine, ribothymidine, 7-methylguanosine, hypoxanthine, and xanthine.

Oligonucleotides further include those comprising entirely or partially of modified nucleobases (semi-synthetically or synthetically derived). See Herdewijn P, Antisense Nucleic Acid Drug Dev 10: 297-310 (2000); and, Sanghvi Y S, et al. Nucleic Acids Res, 21: 3197-3203 (1993).

In some embodiments, at least one nucleoside, i.e., a joined base and sugar, in an oligonucleotide is modified, i.e., a nucleoside mimetic. In certain embodiments, the modified nucleoside comprises a tetrahydropyran nucleoside, wherein a substituted tetrahydropyran ring replaces the naturally occurring pentofuranose ring. See PCT/US2010/022759 and PCT/US2010/023397. In other embodiments, the nucleoside mimetic comprises a 5′-substituent and a 2′-substituent. See PCT/US2009/061913. In some embodiments, the nucleoside mimetic is a substituted α-L-bicyclic nucleoside. See PCT/US2009/058013. In additional embodiments, the nucleoside mimetic comprises a bicyclic sugar moiety. See PCT/US2009/039557. In further embodiments, the nucleoside mimetic comprises a bis modified bicyclic nucleoside. See PCT/US2009/066863. In certain embodiments, the nucleoside mimetic comprises a bicyclic cyclohexyl ring wherein one of the ring carbons is replaced with a heteroatom. See PCT/US2009/033373. In still further embodiments, a 3′ or 5′-terminal bicyclic nucleoside is attached covalently by a neutral internucleoside linkage to the oligonucleotide. See PCT/US2009/039438. In other embodiments, the nucleoside mimetic is a tricyclic nucleoside. See PCT/US2009/037686.

The oligonucleotides of the invention can contain any number of the modifications described herein. The aforementioned modifications may be incorporated uniformly across an entire oligonucleotide, at specific regions or discrete locations within the oligonucleotide including at a single nucleotide. Incorporating these modifications can create chimeric or hybrid oligonucleotides wherein two or more chemically distinct areas exist, each made up of one or more nucleotides.

Oligonucleotides of the invention can be synthesized by any method known in the art, e.g., using enzymatic synthesis and/or chemical synthesis. The oligonucleotides can be synthesized in vitro (e.g., using enzymatic synthesis and chemical synthesis) or in vivo (using recombinant DNA technology well known in the art). In a preferred embodiment, chemical synthesis is used for modified polynucleotides. Chemical synthesis of linear oligonucleotides is well known in the art and can be achieved by solution or solid phase techniques. Preferably, synthesis is by solid phase methods. Automated, solid phase oligonucleotide synthesizers used to construct the oligonucleotides of the invention are available through various vendors including GE Healthcare Biosciences (Piscataway, N.J.).

Oligonucleotide synthesis protocols are well known in the art and can be found, e.g., in U.S. Pat. No. 5,830,653; WO 98/13526; Stec W et al. J Am Chem Soc 106:6077-6079 (1984); Stec W et al. J Org. Chem 50:3908-3913 (1985); Stec W et al. J Chromatog 326:263-280 (1985); LaPlanche J L et al. Nucl Acid Res 26:251-60 (1986); Fasman G D, Practical Handbook of Biochemistry and Molecular Biology (1989). CRC Press, Boca Raton, Fla.; U.S. Pat. Nos. 5,013,830; 5,214,135; 5,525,719; WO 92/03568; U.S. Pat. Nos. 5,276,019; and 5,264,423.

As used herein, the term “antisense oligonucleotide” refers to an oligomeric nucleic acid that is capable of hybridizing with its complementary target nucleic acid sequence resulting in the impairment of the normal function of the target nucleic acid sequence. Antisense oligonucleotides that inhibit CTGF expression have been described and utilized to decrease CTGF expression in various cell types. (See, e.g., PCT/US1996/008140; PCT/US1999/026189; PCT/US1999/029652; PCT/US2002/038618; Kothapalli D et al. Cell Growth Differ 8:61-68, 1997; Shimo T et al. J Biochem (Tokyo) 124:130-140 (1998); Uchio K et al. Wound Repair Regen 12:60-66 (2004); Guha M et al. FASEB J 21:3355-3368 (2007); U.S. Pat. Nos. 6,358,741; 6,965,025; 7,462,602; U.S. Patent Application Publication No. 2008/0070856; U.S. Patent Application Publication No. 2008/0176964; and U.S. Pat. No. 8,802,839; PCT/US02/38618; PCT/US2009/054973; PCT/US2009/054974; PCT/US2009/054975; PCT/US2009/054976; PCT/US2012/023620; and U.S. patent application Ser. No. 13/364,547, incorporated herein by reference in their entirety.

In some embodiments, the oligonucleotides used to decrease the expression of human CTGF mRNA are small interfering RNA (siRNA). As used herein, the terms “small interfering RNA” or “siRNA” refer to single- or double-stranded RNA molecules that induce the RNA interference (RNAi) pathway and act in concert with host proteins, e.g., RNA induced silencing complex (RISC) to degrade mRNA in a sequence-specific fashion. In naturally occurring RNAi, a double-stranded RNA (dsRNA) is cleaved by the RNase III/helicase protein, Dicer, into small interfering RNA (siRNA) molecules. These siRNAs are incorporated into a multicomponent-ribonuclease called RNA-induced silencing complex (RISC). One strand of siRNA remains associated with RISC and guides the complex toward a cognate RNA that has sequence complementary to the guider ss-siRNA in RISC. This siRNA-directed endonuclease digests the RNA, thereby inactivating it.

Selective silencing of CTGF expression by RNAi can be achieved by administering isolated siRNA oligonucleotides or by the in vivo expression of engineered RNA precursors (see U.S. Pat. Nos. 7,056,704, 7,078,196, 7,459,547, 7,691,995 and 7,691,997).

In some embodiments, treatment methods are provided wherein patients are administered a recombinant expression vector that expresses anti-CTGF oligonucleotides. Such genetic constructs can be designed using appropriate vectors and expressional regulators for cell- or tissue-specific expression and constitutive or inducible expression. These genetic constructs can be formulated and administered according to established procedures within the art. In some embodiments, patients are administered recombinant expression vectors that encode a short hairpin oligonucleotide. In further embodiments, the recombinant expression vectors are DNA plasmids, while in other embodiments, the expression vectors are viral vectors. RNA expressing viral vectors can be constructed based on, but not limited to, adeno-associated viruses, retroviruses, adenoviruses, or alphaviruses. In some embodiments, the expression vectors persist in target cells. Alternatively, such vectors can be repeatedly administered as necessary.

In some embodiments, the decrease in expression of CTGF mRNA by an anti-CTGF oligonucleotide comprises the interference in the function of the target CTGF DNA sequence (CTGF gene), typically resulting in decreased replication and/or transcription of the target CTGF DNA. In other embodiments, the decrease in expression of CTGF mRNA by an anti-CTGF oligonucleotide comprises the interference in function of CTGF RNA, typically resulting in impaired splicing of transcribed CTGF RNA (pre-mRNA) to yield mature mRNA species, decreased CTGF RNA stability, decreased translocation of the CTGF mRNA to the site of protein translation and impaired translation of protein from mature mRNA. In other embodiments, the decrease in expression of CTGF mRNA by an anti-CTGF oligonucleotide comprises the decrease in cellular CTGF mRNA number or cellular content of CTGF mRNA. In some embodiments, the decrease in expression of CTGF mRNA by an anti-CTGF oligonucleotide comprises the down-regulation or knockdown of CTGF gene expression. In other embodiments, the decrease in expression of CTGF mRNA by an anti-CTGF oligonucleotide comprises the decrease in CTGF protein expression or cellular CTGF protein content.

In some embodiments, the methods of the invention comprise the administration of an effective amount of an anti-CTGF oligonucleotide that decreases CTGF mRNA transcription rate, cellular CTGF mRNA level, CTGF expression rate, cellular CTGF protein level or interstitial CTGF protein level. In further embodiments, the methods of the invention comprise the administration of an effective amount of an anti-CTGF oligonucleotide that decreases CTGF mRNA transcription rate, cellular CTGF mRNA level, CTGF expression rate, cellular CTGF protein level by at least 5%, at least 10%, at least 15%, at least 20%, at least 25%, at least 30%, at least 35%, at least 40%, at least 45%, at least 50%, at least 60%, at least 70%, at least 80% or at least 90% compared to controls.

### Administration and Dosage

An effective amount of an anti-CTGF agent or pharmaceutical composition thereof can be administered as often as necessary, e.g., once, twice or three times per day, every other day, once, twice, or three times per week, every other week, every three weeks, or monthly. The skilled artisan will appreciate that certain factors may influence the dosage and timing required to effectively treat a subject, including but not limited to the severity or extent of the disease, the administration route, previous treatments, concurrent medications, performance status, weight, gender, race, or ethnicity, and/or age of the subject.

In certain embodiments, the methods for treating ARDS presented herein comprise the administration to a subject in need thereof an anti-CTGF agent at a range from about 0.01 mg to about 10,000 mg, from about 0.1 mg to about 5,000 mg, from about 1.0 mg to about 2,500 mg, from about 1.0 mg to about 1,000 mg, from about 10 mg to about 500 mg, from about 100 mg to about 1,000 mg, from about 0.10 mg to about 50 mg, or from about 0.5 mg to about 50 mg.

In some embodiments, the methods for treating ARDS presented herein comprise the administration to a subject in need thereof at least about 0.1 mg, 0.5 mg, 1.0 mg, 2.0 mg, 4 mg, 8 mg, 16 mg, 25 mg, 50 mg, 100 mg, 200 mg, 400 mg, 800 mg, 1,000 mg, 2,000 mg, 3,000 mg, 5,000 mg, or 10,000 mg of an anti-CTGF agent. In some embodiments, the methods for treating ARDS presented herein comprise the administration to a subject in need thereof not more than about 1 mg, 5 mg, 10 mg, 20 mg, 30 mg, 40 mg, 50 mg, 60 mg, 70 mg, 80 mg, 90 mg, 100 mg, 150 mg, 175 mg, 200 mg, 250 mg, 500 mg, 750 mg, 1,000, 2,000, 5,000 mg, or 10,000 mg of an anti-CTGF agent.

In further embodiments, the methods for treating ARDS presented herein comprise the administration to a subject in need thereof an anti-CTGF agent from about 0.001 mg/kg to about 5,000 mg/kg, about 0.01 mg/kg to about 1000 mg/kg, about 0.1 mg/kg to about 500 mg/kg, or about 1.0 mg/kg to about 100 mg/kg.

In some embodiments, the anti-CTGF agent is administered at appropriate dosages and frequencies over the course of at least about 1 week, 2 weeks, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months 16 months, 20 months, 24 months, 3 years, 4 years or for as long as believed necessary by a healthcare practitioner. In other embodiments, the anti-CTGF agent is administered at appropriate dosages and frequencies for a period of not more than about 1 week, 2 weeks, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months 16 months, 20 months or 24 months, 3 years or 4 years.

In some embodiments, an effective amount of an anti-CTGF antibody is administered at a dose of between about 1 mg/kg to 100 mg/kg, 5 mg/kg to 75 mg/kg, 10 mg/kg to 50 mg/kg, 15 mg/kg to 45 mg/kg, or 20 mg/kg to 60 mg/kg. In other embodiments, an effective amount of the anti-CTGF antibody comprises a dose of about 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 70 mg/kg, or 75 mg/kg. In further embodiments, the anti-CTGF antibody is administered systemically, e.g., i.v. administration. In particular embodiments, the anti-CTGF antibody is administered at 35 mg/kg. In specific embodiments, the anti-CTGF antibody is administered on Day 1, Day 3, Day 7, and Day 14. In some embodiments, Day 1 for antibody dosing equates to Day 1 or Day 2 of subject intubation. In further embodiments, Day 1 for antibody dosing equates to Day 1 or Day 2 of subject experiencing a blood oxygenation level of ≤93%. In other embodiments, the anti-CTGF antibody is administered every 7 days or every 14 days. In other embodiments, the anti-CTGF antibody is pamrevlumab and is administered at 35 mg/kg on Day 1, Day 3, Day 7, and Day 14. In additional embodiments, the anti-CTGF antibody is pamrevlumab and is administered at 35 mg/kg on Day 1, Day 7, Day 14 and Day 28.

In further embodiments, the anti-CTGF antibody is administered at a dosage to achieve a target plasma, or tissue concentration of the anti-CTGF antibody. In particular embodiments, the administered dosage achieves a plasma or tissue target concentration of the anti-CTGF antibody of at least about 10 μg/ml, at least about 50 μg/ml, at least about 100 μg/mL, at least about 200 μg/mL, at least about 300 μg/mL, or at least about 400 μg/mL. In further embodiments, the administration to a subject in need thereof of an anti-CTGF antibody achieves a plasma or tissue target concentration of a range of about 1.0 μg/ml to about 2,000 μg/ml, about 10 μg/mL to about 1,000 μg/mL, or about 20 μg/mL to about 500 μg/mL.

In some embodiments, the anti-CTGF antibody is administered as a loading dose The term “loading dose” as used herein refers to an antibody dose administered to rapidly achieve a desired therapeutic antibody concentration or associated therapeutic effect. Typically, the loading dose is either higher than subsequent doses “maintenance doses” and/or is administered out of sequence, e.g., earlier than other doses. As an example of a higher loading dose, 50 mg/kg of an ant-CTGF antibody is first administered with subsequent doses being 35 mg/kg. As an example of a loading dose being administered out of sequence, if the general administration schedule is every 7 days, i.e., Day 1, Day 7, Day 14, etc., a loading could be administered on Day 3. As a further example, if the general administration schedule is every 14 days, i.e., Day 1, Day 14, Day 28, etc. A loading dose can be administered on Day 7.

In some embodiments, the loading dose is at least 1 mg/kg, 5 mg/kg, 10 mg/kg, 12.5 mg/kg, 15 mg/kg, 20 mg/kg, 22.5 mg/kg, 25 mg/kg, 30 mg/kg, 35 mg/kg, 40 mg/kg, 45 mg/kg, 50 mg/kg, 55 mg/kg, 75 mg/kg, or 100 mg/kg. In other embodiments, the loading dose is the antibody dose that is sufficient to achieve an antibody concentration in blood of at least 0.1 μg/ml, 1.0 μg/ml, 5 μg/ml, 10 μg/ml, 25 μg/ml, 30 μg/ml, 40 μg/ml, 50 μg/ml, 60 μg/ml, 75 μg/ml, 75 μg/ml, 100 μg/ml, 125 μg/ml, 150 μg/m, or 200 μg/ml when measured about 1, 2, 3, 4, 5, 6, 7, or 14 days post-administration (Cₘᵢₙ). In further embodiments, the loading dose is administered on Day 1, Day 2, Day 3, Day 4, Day 5, or Day 6 of a 7 day or 8 day dosing schedule, i.e., Day 1, Day 7, Day 14, etc. In particular embodiments, the loading dose is pamrevlumab, 35 mg/kg, administered on Day 3.

The term “maintenance dose” as used herein refers to an antibody dose sufficient to maintain a desired therapeutic antibody concentration or associated therapeutic effect. For example, a maintenance dose may maintain a reduction in pulmonary exudate achieved with the loading dose. In some embodiments, the maintenance dose is administered about 1, 2, 3, 4, 5, 6, 7, 8, 9, 10, 11, 12, 13, or 14 days post-administration of the loading dose. In other embodiments, the maintenance dose is administered no more than about 1, 2, 3, 4, 5, 6, 7, 8, 9, 10, 11, 12, 13, or 14 days post-administration of the loading dose.

In some embodiments, an effective amount of an anti-CTGF oligonucleotide comprises a dose between about 0.01 mg to about 1,000 mg, about 0.1 mg to about 100 mg, about 1.0 mg to about 50 mg, about 1.0 mg to about 25 mg, or about 5 mg to about 50 mg.

### Combination Therapy

In some embodiments, the methods for treating ARDS provided herein involve the administration of an anti-CTGF agent in combination with one or more additional therapies. As used herein, the term “in combination” refers to the administration of the anti-CTGF agent prior to, concurrent with, or subsequent to the administration of one or more additional therapies for use in treating ARDS or a symptom associated with ARDS. The use of the term “in combination” does not restrict the order in which the anti-CTGF agent and the one or more additional therapies are administered to a subject. The additional therapies may be administered by the same route or a different route of administration than used for the anti-CTGF agent.

In some embodiments, the additional therapy administered in combination with the anti-CTGF agent is an inhibitor of a pro-inflammatory mediator, including pro-inflammatory cytokines and chemokines. In particular embodiments, the inhibitor of pro-inflammatory mediators is an antibody that selectively binds to the pro-inflammatory mediator or to a receptor for the pro-inflammatory mediator. In other embodiments, the inhibitor is a small molecule. In further embodiments, the inhibitor is an inhibitor of interleukin-1 (IL-1), interleukin-6 (IL-6), interleukin-12 (IL-12), interleukin-18 (IL-18), interferon gamma (INF-γ), granulocyte-macrophage colony stimulating factor (GM-CSF), tumor necrosis factor alpha (TNF-α), or the respective receptors thereof.

In additional embodiments, the IL-1 inhibitor is the IL-1 receptor antagonist anakinra, the soluble decoy receptor rilonacept and the neutralizing monoclonal anti-IL-1β antibody canakinumab. In particular embodiments, the IL-6 inhibitor is an antibody to IL-6 including Olokizumab, Clazakizumab or Sirukumab. In other embodiments, the IL-6 inhibitor is an antibody to the interleukin-6 receptor (IL-6R). In specific embodiments, the anti-IL-6R antibody is Tocilizumab, also known as atlizumab, or Sarilumab, trade name Kevzara.

In further embodiments, the IL-12 inhibitor is an antibody to IL-12 including ustekinumab, trade name Stelara, and briakinumab (ABT-874). In some embodiments, the IFNγ inhibitor is an antibody including emapalumab, trade name Gamifant. In other embodiments, the GM-CSF inhibitor is an antibody, including gimsilumab.

In some embodiments, the anti-CTGF agent is administered in combination with an agent that inhibits a form of vascular endothelial growth factor (VEGF), for example, VEGF-A, or its receptor. In particular embodiments, the VEGF inhibitor is bevacizumab, trade name Avastin.

In additional embodiments, the anti-CTGF agent is administered in combination with an agent that inhibits the production of reactive oxygen species (ROS) or reactive nitrogen species (RNS). In further embodiments, the anti-CTGF agent is administered in combination with an anti-oxidant agent or an agent that neutralizes ROS or RNS. In particular embodiments, the anti-oxidant or neutralizing agent is vitamin C, glutathione, or n-acetyl-L-cysteine (NAC).

In some embodiments, the anti-CTGF agent is administered in combination with an anti-inflammatory agent including corticosteroids or non-steroidal anti-inflammatory drugs (NSAIDs). In specific embodiments, the corticosteroid is dexamethasone or methylprednisolone.

In some embodiments, the anti-CTGF agent is administered in combination with a is an inhibitor of janus kinase (JAK). In specific embodiments, the JAK inhibitor is baricitinib, trade name Olumiant.

In some embodiments, the anti-CTGF agent is administered in combination with an angiotensin receptor blocker. In specific embodiments, the angiotensin receptor blocker is losartan. In other embodiments, the anti-CTGF agent is administered in combination with chloroquine, hydroxychloroquine, azithromycin, a histamine lantagonist, histamine 2 antagonist, and/or a proton pump inhibitor. In further embodiments, the anti-CTGF agent is administered in combination with an antiviral medication, e.g., protease inhibitors, influenza drugs, nucleoside analogs, including lopinavir, ritonavir, remdesivir, oseltamivir, or favipiravir. In additional embodiments, the anti-CTGF agent is administered in combination with stem cell therapy, e.g., MultiStem. In other embodiments, the anti-CTGF agent is administered in combination with immunoglobulin or thymosin.

In further embodiments, the anti-CTGF agent is administered in combination with oxygen or ventilatory assistance, e.g., intermittent positive pressure ventilation, bilevel positive airway pressure, or biphasic cuirass ventilation, or inhaled nitrous oxide.

In specific embodiments, the interval of time between the administration of an anti-CTGF agent and the administration of one or more additional therapies may be about 0 to 15 minutes, 0 to 30 minutes, 30 minutes to 60 minutes, 1 to 2 hours, 2 to 6 hours, 2 to 12 hours, 12 to 24 hours, 1 to 2 days, 2 to 4 days, 4 to 7 days, 1 to 2 weeks, or 2 to 4 weeks. In certain embodiments, the anti-CTGF agent and one or more additional therapies are administered less than 30 minutes, 1 hour, 2 hours, 3 hours, 4 hours, 5 hours, 6 hours, 7 hours, 8 hours, 9 hours, 10 hours, 12 hours, 16 hours, 20 hours, 1 day, 2 days, 3 days, 4 days, 5 days, 6 days, 1 week, or 2 weeks apart.

In some embodiments, the administration of an anti-CTGF agent in combination with one or more additional therapies has an additive effect, while in other embodiments, the combination of therapies have a synergistic effect. In specific embodiments, a synergistic effect achieved with combination therapy permits the use of lower dosages (e.g., sub-optimal conventional doses) of the additional therapy, e.g., a corticosteroid. In other embodiments, the synergistic effect achieved with combination therapy allows for a less frequent administration of the additional therapy to a subject. In certain embodiments, the ability to utilize lower dosages of an additional therapy and/or to administer the additional therapy less frequently reduces the toxicity associated with the administration of the additional therapy, without reducing the efficacy of the additional therapy. In some embodiments, a synergistic effect results in improved efficacy of an anti-CTGF antibody and/or the additional therapies in treating ARDS.

The combination of an anti-CTGF agent and one or more additional therapies can be administered to a subject in the same pharmaceutical composition. Alternatively, an anti-CTGF agent and one or more additional therapies can be administered to a subject in separate pharmaceutical compositions. An anti-CTGF agent and one or more additional therapies may also be administered to a subject by the same or different routes of administration.

### Pharmaceutical Formulations and Routes of Administration

The compositions and compounds suitable for use in the methods of the present invention can be delivered directly or in pharmaceutical compositions containing excipients, as is well known in the art. Various formulations and drug delivery systems are available in the art and depend in part on the intended route of administration. (See, e.g., Gennaro A R, ed. Remington's Pharmaceutical Sciences, (2000); and Hardman J G, Limbird L E, and Gilman L S, eds. The Pharmacological Basis of Therapeutics, (2001))

Suitable routes of administration may, for example, include oral, rectal, topical, nasal, pulmonary, intestinal, and parenteral administration. Primary routes for parenteral administration include intravenous, intramuscular, and subcutaneous administration. Secondary routes of administration include intraperitoneal, and intra-arterial administration.

Pharmaceutical dosage forms of a suitable compound for use in the invention may be provided in an instant release, controlled release, sustained release, or target drug-delivery system. Commonly used dosage forms include, for example, solutions and suspensions, (micro-) emulsions, ointments, gels and patches, liposomes, tablets, dragees, soft or hard shell capsules, suppositories, ovules, implants, amorphous or crystalline powders, aerosols, and lyophilized formulations. Depending on route of administration used, special devices may be required for application or administration of the drug, such as, for example, syringes and needles, inhalers, pumps, injection pens, applicators, or special flasks. Pharmaceutical dosage forms are often composed of the drug, an excipient(s), and a container/closure system. One or multiple excipients, also referred to as inactive ingredients, can be added to a compound of the invention to improve or facilitate manufacturing, stability, administration, and safety of the drug, and can provide a means to achieve a desired drug release profile. Therefore, the type of excipient(s) to be added to the drug can depend on various factors, such as, for example, the physical and chemical properties of the drug, the route of administration, and the manufacturing procedure. Pharmaceutically acceptable excipients are available in the art, and include those listed in various pharmacopoeias. (See, e.g., USP, JP, EP, and BP), Inactive Ingredient Guide available through the FDA's website, and Handbook of Pharmaceutical Additives, ed. Ash; Synapse Information Resources, Inc. (2002))

Pharmaceutical dosage forms of a compound for use in the present invention may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes. As noted above, the compositions for use in the present invention can include one or more physiologically acceptable inactive ingredients that facilitate processing of active molecules into preparations for pharmaceutical use.

Proper formulation is dependent upon the desired route of administration. For intravenous injection, for example, the composition may be formulated in aqueous solution, if necessary, using physiologically compatible buffers, including, for example, phosphate, histidine, or citrate for adjustment of the formulation pH, and a tonicity agent, such as, for example, sodium chloride or dextrose. For transmucosal or nasal administration, semisolid, liquid formulations, or patches may be preferred, possibly containing penetration enhancers. Such penetrants are generally known in the art. For oral administration, the compounds can be formulated in liquid or solid dosage forms and as instant or controlled/sustained release formulations. Suitable dosage forms for oral ingestion by a subject include tablets, pills, dragees, hard and soft shell capsules, liquids, gels, syrups, slurries, suspensions, and emulsions.

Solid oral dosage forms can be obtained using excipients, which may include, fillers, disintegrants, binders (dry and wet), dissolution retardants, lubricants, glidants, antiadherants, cationic exchange resins, wetting agents, antioxidants, preservatives, coloring, and flavoring agents. These excipients can be of synthetic or natural source. Examples of such excipients include cellulose derivatives, citric acid, dicalcium phosphate, gelatine, magnesium carbonate, magnesium/sodium lauryl sulfate, mannitol, polyethylene glycol, polyvinyl pyrrolidone, silicates, silicium dioxide, sodium benzoate, sorbitol, starches, stearic acid or a salt thereof, sugars (i.e. dextrose, sucrose, lactose, etc.), talc, tragacanth mucilage, vegetable oils (hydrogenated), and waxes. Ethanol and water may serve as granulation aides. In certain instances, coating of tablets with, for example, a taste-masking film, a stomach acid resistant film, or a release-retarding film is desirable. Natural and synthetic polymers, in combination with colorants, sugars, and organic solvents or water, are often used to coat tablets, resulting in dragees. When a capsule is preferred over a tablet, the drug powder, suspension, or solution thereof can be delivered in a compatible hard or soft shell capsule.

Compositions formulated for parenteral administration by injection are usually sterile and, can be presented in unit dosage forms, e.g., in ampoules, syringes, injection pens, or in multi-dose containers, the latter usually containing a preservative. The compositions may take such forms as suspensions, solutions, or emulsions in oily or aqueous vehicles, and may contain formulatory agents, such as buffers, tonicity agents, viscosity enhancing agents, surfactants, suspending and dispersing agents, antioxidants, biocompatible polymers, chelating agents, and preservatives. Depending on the injection site, the vehicle may contain water, a synthetic or vegetable oil, and/or organic co-solvents. In certain instances, such as with a lyophilized product or a concentrate, the parenteral formulation would be reconstituted or diluted prior to administration. Depot formulations, providing controlled or sustained release of a compound of the invention, may include injectable suspensions of nano/micro particles or nano/micro or non-micronized crystals. Polymers such as poly(lactic acid), poly(glycolic acid), or copolymers thereof, can serve as controlled/sustained release matrices, in addition to others well known in the art. Other depot delivery systems may be presented in form of implants and pumps requiring incision.

Suitable carriers for intravenous injection for the molecules of the invention are well-known in the art and include water-based solutions containing a base, such as, for example, sodium hydroxide, to form an ionized compound, sucrose or sodium chloride as a tonicity agent, for example, the buffer contains phosphate or histidine. Co-solvents, such as, for example, polyethylene glycols, may be added. These water-based systems are effective at dissolving compounds of the invention and produce low toxicity upon systemic administration. The proportions of the components of a solution system may be varied considerably, without destroying solubility and toxicity characteristics. Furthermore, the identity of the components may be varied. For example, low-toxicity surfactants, such as polysorbates or poloxamers, may be used, as can polyethylene glycol or other co-solvents, biocompatible polymers such as polyvinyl pyrrolidone may be added, and other sugars and polyols may substitute for dextrose.

Anti-CTGF antibody formulations for use in accordance with the present invention may be prepared by mixing an anti-CTGF antibody with pharmaceutically acceptable carriers, excipients or stabilizers that are nontoxic to recipients at the dosages and concentrations employed. Anti-CTGF antibody formulations may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); carriers; hydrophilic polymers such as polyvinylpyrrolidone; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; and/or non-ionic surfactants or polyethylene glycol.

In particular, anti-CTGF antibody formulations may further comprise low molecular weight polypeptides; carriers such as serum albumin, gelatin, or immunoglobulins; and amino acids such as glycine, glutamine, asparagine, histidine, arginine, or lysine. The anti-CTGF antibody formulations can be lyophilized as described in PCT/US1996/012251. Additionally, sustained-release preparations may also be prepared. Frequently, polymers such as poly(lactic acid), poly(glycolic acid), or copolymers thereof serve as controlled/sustained release matrices, in addition to others well known in the art.

The anti-CTGF antibodies can be supplied or administered at any desired concentration. In some embodiments, the anti-CTGF antibody concentration is at least 1 mg/ml, 5 mg/ml, 10 mg/ml, 20 mg/ml, 25 mg/ml, 50 mg/ml, 75 mg/ml, 100 mg/ml, 125 mg/ml, 150 mg/ml, or 200 mg/ml. In other embodiments, the anti-CTGF antibody concentration is no more than about 5 mg/ml, 10 mg/ml, 20 mg/ml, 25 mg/ml, 50 mg/ml, 75 mg/ml, 100 mg/ml, 125 mg/ml, 150 mg/ml, 200 mg/ml, 250 mg/ml, or 300 mg/ml. In further embodiments, the anti-CTGF antibody concentration is between 5 mg/ml to 20 mg/ml, 20 mg/ml to 50 mg/ml, 50 mg/ml to 100 mg/ml, 100 mg/ml to 200 mg/ml, or 200 mg/ml to 300 mg/ml.

### Articles of Manufacture

The present compositions may, if desired, be presented in a pack or dispenser device containing one or more unit dosage forms containing an anti-CTGF agent. Such a pack or device may, for example, comprise metal or plastic foil, glass and rubber stoppers, vials or syringes. The container holding the anti-CTGF agent composition may have a sterile access port (for example the container may be an intravenous solution bag or a vial having a stopper pierceable by a hypodermic injection needle). The article of manufacture may further comprise an additional container comprising a pharmaceutically acceptable diluent buffer, such as bacteriostatic water for injection (BWFI), phosphate-buffered saline, Ringer's solution, and/or dextrose solution. The article of manufacture may further include other materials desirable from a commercial and user standpoint, including, for example, filters or needles.

Compositions comprising an anti-CTGF agent formulated in a compatible pharmaceutical carrier may be provided in an appropriate container that is labeled for treatment of ARDS. The pack or dispenser device may be accompanied by instructions for administration that provide specific guidance regarding dosing the anti-CTGF agent including a description of the type of patients who may be treated (e.g., a person at risk of developing ARDS), the schedule (e.g., dose and frequency) and route of administration, and the like.

These and other embodiments of the present invention will readily occur to those of ordinary skill in the art in view of the disclosure herein.

### EXAMPLES

The invention will be further understood by reference to the following examples, which are intended to be purely exemplary of the invention. These examples are provided solely to illustrate the claimed invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications are intended to fall within the scope of the appended claims.

### Example 1: An Open-Label Phase II/III Study of Anti-CTGF Monoclonal Antibody Pamrevlumab in Patients with COVID-19-Associated ARDS

The efficacy and safety of intravenous administration of pamrevlumab versus standard of care in patients with SARS-CoV-2 infection is measured by administering an effective amount of the anti-CTGF therapeutic agent pamrevlumab to patients requiring mechanical ventilation. In general, patients with documented SARS-CoV-2 infection, age >20 to <85 years with interstitial pneumonia (as assessed by chest X-ray or HRCT) and respiratory distress requiring hospitalization are randomized to receive treatment with pamrevlumab in addition to standard of care or just standard of care in a 1:1 ratio. Standard of care may include background therapy according to standard clinical practice, including antimicrobial, prophylactic, and best supportive care therapies as deemed appropriate by the Investigator. Analgesic treatment, transfusion of blood products, electrolyte and glucose infusions, IV parenteral nutrition, inotropic support, antibiotics, anti-fungal and anti-viral treatments, ultrafiltration, or hemodialysis, as well as general supportive care are permitted.

Patients assigned to the pamrevlumab arm are dosed with pamrevlumab at 35 mg/kg IV administered at Day 1, Day 3, Day 7, and Day 14. Patients will be assessed for various clinical parameters according to the schedule listed in Table 1. In a subgroup of patients, and based on the investigator's decision, treatment is continued every 2 weeks after Day 14, up to 12 weeks.

TABLE 1
Clinical Trial Assessment Schedule

| | | | | | | Follow- | Follow- |
|---|---|---|---|---|---|---|---|
| | Screening | | | | | up | up |
| | Up to 72 | | | | | Day 21 | Day 28 |
| | hrs prior | Day 1 | | | | (±2 | (±4 |
| ASSESSMENT | to Day 1 | (baseline) | Day 3 | Day 7 | Day 14 | days) | days) |
| Informed consent | X | | | | | | |
| Eligibility criteria | X | X | | | | | |
| Demographic | X | | | | | | |
| information | | | | | | | |
| Physical | X | | | | | | |
| examination | | | | | | | |
| Vital signs | X | X | | X | X | | |
| assessment | | | | | | | |
| ECG assessment | X | | | | | | |
| HRCT scan of | X | | | | X | | |
| chest | | | | | | | |
| Laboratory | X | X | | X | X | | |
| assessments (local) | | | | | | | |
| Urine pregnancy | X | | | | | | |
| test | | | | | | | |
| Randomization | | X | | | | | |
| Concomitant | | X | | X | X | | |
| medication | | | | | | | |
| (including | | | | | | | |
| background | | | | | | | |
| therapy) | | | | | | | |
| PaO₂/FiO₂ | X | X | | X | X | | |
| Resting SpO₂ (3 | | X | | X | X | | |
| times/day) | | | | | | | |
| Hemogasanalysis | | X | | X | X | | |
| Oxygen | | X | | X | X | | |
| supplementation | | | | | | | |
| Adverse events | | X | | X | X | | |
| Biomarkers | | X | | X | X | | |
| Survival | | | | | | X | X |
| Hospitalization | | | | | | X | X |
| days and discharge | | | | | | | |

The effect of pamrevlumab on the efficiency of oxygenation in patients with SARS-CoV-2 infection requiring hospitalization is assessed, as well as the long-term effect of pamrevlumab on lung function. The primary endpoint is change from baseline in PaO₂/FiO₂ at Day 5. The key secondary endpoint is number of days not requiring invasive mechanical ventilation up to Day 15. Other secondary endpoints include time on ventilator, time from start of study to requiring mechanical ventilation, change from baseline to day 5 in resting SpO₂, change from baseline to Day 5 in oxygen supplementation, change from baseline in chest high-resolution CT (HRCT) scan to Day 15 (subgroup of enrolled patients), time to hospital discharge, and time to all-cause mortality. A reduction in any of these endpoints indicates that the anti-CTGF agent is beneficial in patients having ARDS.

### Example 2: A Randomized, Double Blinded, Placebo-Controlled Phase II Study of Anti-CTGF Monoclonal Antibody Pamrevlumab in Hospitalized Patients with COVID-19-Associated ARDS

The efficacy and safety of intravenous administration of pamrevlumab versus standard of care in patients with SARS-CoV-2 infection is measured by administering an effective amount of an anti-CTGF therapeutic agent, pamrevlumab, to patients with documented SARS-CoV-2 infection, age 40 to 85 years, with evidence of respiratory compromise requiring hospital admission. In general, approximately 130 patients are randomized in a 1:1 ratio to either pamrevlumab or placebo (control); all patients also receive standard-of-care in the judgment of the Investigator. Pamrevlumab is administered via IV infusion on Days 1 (day of randomization), 7, 14, and 28. The clinical status of patients is assessed daily, until Day 28, using an 8-point Modified WHO Ordinal Scale. The screening, treatment, and clinical assessment schedule is shown in Table 2.

TABLE 2
Clinical Trial Assessment Schedule

| | Screening | | | | | | | | |
|---|---|---|---|---|---|---|---|---|---|
| | Up to 2 | | | | | | | | 4-Weeks |
| | days prior | Day 1 | Days | | Days | | Days | | after last |
| ASSESSMENT | to Day 1 | (baseline) | 2-6 | Day 7 | 8-13 | Day 14 | 15-27 | Day 28 | dose |
| Informed | X | | | | | | | | |
| consent | | | | | | | | | |
| Eligibility | X | | | | | | | | |
| criteria | | | | | | | | | |
| Medical history | X | | | | | | | | |
| Demographic | X | | | | | | | | |
| information | | | | | | | | | |
| Vital signs | X | X | X | X | X | X | X | X | |
| assessment | | | | | | | | | |
| Urine pregnancy | X | | | | | | | | |
| test | | | | | | | | | |
| Concomitant | X | X | X | X | X | X | X | X | X |
| medication and | | | | | | | | | |
| non-drug | | | | | | | | | |
| therapies | | | | | | | | | |
| Infusion | | X | | X | | X | | X | |
| Local laboratory | | X | | X | | X | | X | X |
| tests | | | | | | | | | |
| (hematology: | | | | | | | | | |
| CBC and | | | | | | | | | |
| differential, | | | | | | | | | |
| chemistry: CMP, | | | | | | | | | |
| coagulation | | | | | | | | | |
| parameters: INR, | | | | | | | | | |
| PT, aPTT) | | | | | | | | | |
| Arterial blood | X | X | X | X | X | X | X | X | |
| gasses | | | | | | | | | |
| Resting SpO2 | X | X | X | X | X | X | X | X | |
| Oxygen | X | X | X | X | X | X | X | X | |
| supplementation | | | | | | | | | |
| needs | | | | | | | | | |
| WHO ordinal | | X | X | X | X | X | X | X | |
| scale | | | | | | | | | |
| Adverse events | X | X | X | X | X | X | X | X | X |
| Survival, ICU, | | X | X | X | X | X | X | X | X |
| and hospital | | | | | | | | | |
| discharge status | | | | | | | | | |

Evidence that treatment with an anti-CTGF agent is beneficial for patients with ARDS includes an increase, compared to placebo treated patients, in: 1) the proportion of patients who never receive mechanical ventilation and/or ECMO and are alive at Day 28; 2) the proportion of patients that are alive, discharged home, and not on supplemental oxygen at Day 28; 3) the proportion of patients who never receive mechanical ventilation and/or ECMO and alive at Day 14; or 4) the time to death from any cause at Day 28.

Further evidence that treatment with an anti-CTGF agent is beneficial for patients with ARDS includes a reduction, compared to placebo treated patients, in 1) the number of days patients spend in ICU/CCU (either on or off mechanical ventilation and/or ECMO) assessed at Day 28; 2) the number of days patients spend on mechanical ventilation and/or ECMO assessed on Day 28; or 3) all-cause mortality at Day 28 (proportion of patients deceased).

Additionally, evidence of benefit from treatment with an anti-CTGF agent include, compared to placebo treated patients: 1) an improvement, since initiation of treatment (Day 1), in ARDS categorization, using the Berlin or other suitable criteria 2) an improvement, since initiation of treatment, in blood oxygenation or 3) a reduction, since initiation of treatment, in (non-invasive) oxygen supplementation requirements.

Other evidence of beneficial improvement associated with treatment with an anti-CTGF agent include, compared to placebo treated patients, a reduction in recovery time (by Day 28).

Various modifications of the invention, in addition to those shown and described herein, will become apparent to those skilled in the art from the foregoing description. Such modifications are intended to fall within the scope of the appended claims.

All references cited herein are hereby incorporated by reference herein in their entirety.

What is claimed is:

1. A method of improving, compared to a baseline measurement, the blood oxygenation level of a subject diagnosed with COVID-19, the method comprising administering to the subject an effective amount of an anti-CTGF agent, thereby improving the subject's blood oxygenation level.

2. The method of claim 1, wherein the subject is further diagnosed with acute respiratory distress syndrome (ARDS).

3. The method of claim 1, wherein the blood oxygenation level is determined using the oxygenation index, the oxygenation saturation index, or pulse oximetry.

4. A method of preventing the development of ARDS, or reducing the severity of at least one ARDS-associated symptom or clinical parameter in a subject in need thereof, the method comprising administering to the subject an effective amount of an anti-CTGF agent, thereby preventing the development of ARDS, or reducing the severity of at least one ARDS-associated symptom or clinical parameter in the subject.

5. A method of treating ARDS, the method comprising administering to a subject in need thereof an effective amount of an anti-CTGF agent, thereby treating the subject's ARDS.

6. The method of claim 5, wherein the subject is infected with an infectious agent.

7. The method of claim 5, wherein the subject suffered a pulmonary injury from a non-infectious agent.

8. The method of claim 6, wherein the infectious agent is a virus.

9. The method of claim 8, wherein the virus is a coronavirus selected from the group consisting of SARS-CoV, HCoV NL63, HKU1, MERS-CoV, and SARS-CoV-2.

10. The method of claim 9, wherein the coronavirus is SARS-CoV-2.

11. The method of claim 5, wherein treatment with an anti-CTGF agent reduces ventilator use, reduces ECMO use, reduces time in the ICU or CCU, reduces hospitalization time, reduces all-cause mortality, or reduces supplemental oxygen consumption compared to a control group or historical controls.

12. The method of claim 5, wherein treatment with an anti-CTGF agent reduces pulmonary exudate levels or reduces supplemental oxygen consumption compared to a baseline measurement.

13. The method of claim 5, wherein treatment with an anti-CTGF agent increases subject's survival compared to a control group or historical controls.

14. The method of claim 5, wherein the anti-CTGF agent is selected from group consisting of anti-CTGF antibodies, anti-CTGF antibody fragments, anti-CTGF antibody mimetics, and anti-CTGF oligonucleotides.

15. The method of claim 13, wherein the anti-CTGF agent is an anti-CTGF antibody.

16. The method of claim 14, wherein the anti-CTGF antibody is a human or humanized antibody.

17. The method of claim 14, wherein the anti-CTGF antibody is pamrevlumab.

18. The method of claim 5, wherein the effective amount of an anti-CTGF antibody is at least 35 mg/kg.

19. The method of claim 16, wherein pamrevlumab is administered at 35 mg/kg on Day 1 and Day 3.

20. The method of claim 16, wherein pamrevlumab is administered at 35 mg/kg on Day 1 and Day 7.

21. The method claim 13, wherein the anti-CTGF oligonucleotide is selected from the group consisting of antisense oligonucleotides, siRNAs, miRNAs and shRNAs.

22. The method of claim 5, further comprising the administration of an inhibitor of a pro-inflammatory mediator, an inhibitor of VEGF, or an anti-oxidant.
